(19) United States
(12) Patent Application Publication
(10) Pub. No.: US 2012/0045500 A1
(43) Pub. Date: Feb. 23, 2012

(54) NOVEL DIOXO-IMIDAZOLIDINE DERIVATIVES, WHICH INHIBIT THE ENZYME SOAT-1, AND PHARMACEUTICAL AND COSMETIC COMPOSITIONS CONTAINING THEM

(75) Inventors: Cédric Poinsard, Le Plan de Grasse (FR); Thibaud Portal, Opio (FR); Jean-Claude Pascal, Nice (FR)

(73) Assignee: GALDERMA RESEARCH & DEVELOPMENT, Biot (FR)

(21) Appl. No.: 13/203,169

(22) PCT Filed: Feb. 26, 2010
(86) PCT No.: PCT/EP2010/052500

§ 371 Date: Nov. 8, 2011

Related U.S. Application Data
(60) Provisional application No. 61/202,415, filed on Feb. 26, 2009.

(30) Foreign Application Priority Data
Jun. 5, 2009 (FR) 0953756

(51) Int. Cl. A61K 31/438; C07D 235/02; A61K 31/4184; A61K 9/127; A61K 9/70; A61P 27/02; A61P 7/00; A61P 9/10; A61P 25/28; A61P 17/10; A61P 17/08; C07D 471/10; A61P 17/00; B82Y 5/00

(52) U.S. Cl. 424/443; 546/20; 514/278; 548/301.4; 514/389; 424/450; 977/773; 977/915

(57) ABSTRACT

Compounds of general formula (I):

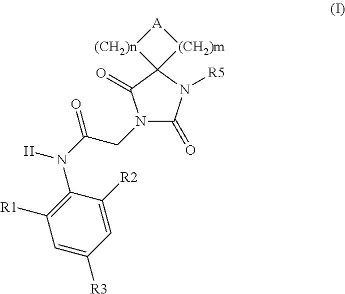

and cosmetic and pharmaceutical compositions including such a compound are described.

The invention relates to novel dioxo-imidazolidine derivatives, which are inhibitors of the enzyme SOAT-1 (Sterol-O-Acyl Transferase-1, also known as ACAT-1: Acyl-coenzyme A Cholesterol Acyl Transferase). The invention also relates to the use of these derivatives in pharmaceutical compositions intended for use in human or veterinary medicine, or alternatively in cosmetic compositions, and also to their non-therapeutic use.

Compositions with activity of SOAT-1-inhibiting type are widely described in the literature as having activity in regulating biological processes involving cholesterol and derivatives thereof. These properties give this class of compounds strong potential in the treatment or prevention of many pathologies, and more particularly in dermatology and in cardiovascular diseases or central nervous system complaints. Most of the biological effects of SOAT-1 inhibitors are mediated by prevention of the synthesis of cholesterol esters by the enzyme SOAT-1. Among the prior art documents describing SOAT-1-inhibiting molecules, mention may be made, for example, of WO 96/10559, EP 0 370 740, EP 0 424 194, U.S. Pat. No. 4,623,663, EP 0 557 171, U.S. Pat. No. 5,003,106, EP 0 293 880, EP 0 433 662 and U.S. Pat. No. 5,106,873, which describe compounds for treating arteriosclerosis or hypercholesterolaemia. The therapeutic potential of SOAT-1 inhibitors in the treatment of cardiovascular diseases, and in particular of hypercholesterolaemia and arteriosclerosis, is also described by Kharbanda R. K. et al., in Circulation, 2005, 11, 804. The potential of SOAT-1 inhibitors for the treatment of Alzheimer's disease has also been reported in the literature, for example by Puglielli, L. et al., in Nature Neurosciences 2003, 6 (4), 345.

U.S. Pat. No. 6,133,326, U.S. Pat. No. 6,271,268 and WO 2005/034 931 describe SOAT-1-inhibiting compounds for inhibiting the production of sebum. In the field of dermatology, in particular, it is particularly advantageous to prevent excessive sebum production and all the associated conditions. Sebum is produced by the sebaceous glands. The largest concentration of sebaceous glands is found on the face, the shoulders, the back and the scalp. Sebum is secreted at the surface of the skin, where it plays a major physiological role, associated with maintaining the skin barrier and a microenvironment that permits regulation of the cutaneous bacterial and fungal flora.

Sebum hyperproduction is usually associated with a skin or scalp of greasy appearance, which is a cause of discomfort and of degraded appearance. Moreover, sebum hyperproduction may give rise to seborrhoeic dermatitis and is associated with an increased incidence or worsening of acne. The cholesterol esters produced in the sebaceous glands by SOAT-1 are one of the components of sebum, among several classes of lipids including triglycerides, wax esters and squalenes, as described by Nikkari, T., in J. Invest. Derm. 1974, 62, 257. Inhibition of this enzyme or of other acyl transferases may thus make it possible to inhibit sebum production. U.S. Pat. No. 6,133,326 especially describes the inhibition of sebum with ACAT-1 (also known as SOAT-1) inhibitors. However, at the present time, no treatment using such inhibitors is commercially available. The only treatments that can remedy or relieve hyperseborrhoea-related disorders are systemic hormonal treatments or systemic treatment with 13-cis-retinoic acid, the side effects of which treatments greatly limit their field of application. There is thus a clear medical and cosmetic need to treat complaints and pathologies related to sebum hyperproduction.

In this context, the present invention proposes to provide novel dioxo-imidazolidine derivatives that are powerful inhibitors of the enzyme SOAT-1.

One subject of the invention is novel dioxo-imidazolidine derivatives, which are inhibitors of the enzyme SOAT-1, and which correspond to the general formula (I) below:

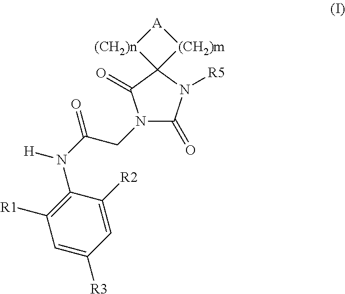

in which:
R₁ represents a group C₂₋₆ alkyl, C₃₋₇ cycloalkyl, C₁₋₆ alkyloxy, C₁₋₆ fluoroalkyl, C₁₋₆ fluoroalkyloxy or a group —(C₁₋₁₂)ₙ—C₃₋₇ cycloalkyl,
R₂ and R₃ are identical or different and represent a hydrogen, chlorine, fluorine, bromine or iodine atom or a group C₁₋₆ alkyl, C₃₋₆ cycloalkyl, C₂₋₆ alkylthio, C₁₋₆ alkyloxy, C₁₋₆ fluoroalkyl, C₁₋₆ fluoroalkyloxy or a group —(C₁₋₁₂)ₙ—C₃₋₇ cycloalkyl,
R₅ represents a group chosen from:
   an unsubstituted phenyl group or a phenyl group substituted with one, two or three identical or different substituents chosen from fluorine, chlorine and bromine atoms and groups C₁₋₄ alkyl, C₁₋₄ alkylthio, trifluoromethyl, hydroxymethyl, mono-, di- and tri-fluoromethoxy, C₁₋₄ alkyloxy, phenoxy, benzyloxy, phenyl, 2-pyridyl, 3-pyridyl and 4-pyridyl,
   a linear or branched group C₂₋₁₂ alkyl, optionally substituted with one or more hydroxyl groups or fluorine atoms,
   a group C₃₋₁₂ cycloalkyl or a group —(CH₂)ₚ—C₂₋₁₂ cycloalkyl,
   a group —(CH₂)ₙ-aryl in which n is equal to 1, 2 or 3 and the aryl group may be optionally substituted with one or more groups Rₐ,
A represents:
   either an oxygen atom,
   or a group S(O)p,
   or a group NR_{b},
   or a carbon atom substituted with one or two fluorine atoms or a hydroxyl group,
R_{b} represents the following groups: H, C₁₋₆ alkyl, C(O)C₁₋₆ alkyl, C(O)aryl, C(O)—(CH₂)ₚ-aryl, the aryl groups mentioned possibly being optionally substituted with one or more groups Rₐ,
Rₐ represents either a hydrogen, fluorine or chlorine atom or a group C₁₋₆ alkyl, C₃₋₇ cycloalkyl, C₁₋₆ alkyloxy, C₁₋₆ alkylthio, C₁₋₆ fluoroalkyl, C₁₋₆ fluoroalkyloxy, OH, CH₂OH, COORc or CN,
R_{c} represents a group C₁₋₆ alkyl, C₃₋₇ cycloalkyl or —(CH₂)ₙ—C₃₋₇ cycloalkyl,
m and n each represents an integer, the sum of which possibly ranges from 3 to 6,
p represents 0, 1 or 2,
   and also the pharmaceutically acceptable salts, solvates or hydrates thereof and the conformers or rotamers thereof.

The compounds of formula (I) may comprise one or more asymmetric carbon atoms. They may thus exist in the form of a mixture of enantiomers or of diastereoisomers. These enantiomers and diastereoisomers, and also mixtures thereof, including racemic mixtures, form part of the invention.

The compounds of formula (I) may exist in the form of bases or of acid-addition salts. Such addition salts form part of the invention. These salts are advantageously prepared with pharmaceutically acceptable acids, but the salts of other acids that are useful, for example for purifying or isolating the compounds of formula (I), also form part of the invention. These acids may be, for example, picric acid, oxalic acid or an optically active acid, for example a tartaric acid, a dibenzoyltartaric acid, a mandelic acid or a camphorsulfonic acid, and those that form physiologically acceptable salts, such as hydrochloride, hydrobromide, sulfate, hydrogen sulfate, dihydrogen phosphate, maleate, fumarate, 2-naphthalenesulfonate or para-toluenesulfonate. For a review of physiologically acceptable salts, see the Handbook of Pharmaceutical Salts: Properties, Selection and Use by Stahl and Wermuth (Wiley-VCH, 2002).

The solvates or hydrates may be obtained directly after the synthetic process, compound (1) being isolated in the form of a hydrate, for example a monohydrate or hemihydrate, or of a solvate of the reaction or purification solvent.

The present invention includes the isotopically labelled pharmaceutically acceptable compounds of formula (I) in which one or more atoms are replaced with atoms having the same atomic number but an atomic mass or a mass number different from the atomic mass or the mass number that naturally predominates. Examples of isotopes that may be included in the compounds of the invention include hydrogen isotopes such as ²H and ³H, carbon isotopes such as ¹¹C, ¹³C and ¹⁴C, chlorine isotopes such as ³⁶Cl, fluorine isotopes such as ¹⁸F, iodine isotopes such as ¹²³I and ¹²⁵I, nitrogen isotopes such as ¹³N and ¹⁵N, oxygen isotopes such as ¹⁵O, ¹⁷O and ¹⁸O, phosphorus isotopes such as ³²P and sulfur isotopes such as ³⁵S. Substitutions with isotopes that emit positrons, such as ¹¹C, ¹⁸F, ¹⁵O and ¹³N, may be useful in Positron Emission Tomography studies for studying the occupation of receptors.

In the context of the invention, the following definitions apply:
aryl: a monocyclic or bicyclic aromatic group containing 6 to 10 carbon atoms. Examples of aryl groups that may be mentioned include phenyl and naphthyl groups,
C_{b-c} in which b and c may take values from 1 to 6, a carbon-based chain of b to c carbon atoms, for example C₁₋₆ is a carbon-based chain that may contain from 1 to 6 carbon atoms,
alkyl: a linear or branched saturated aliphatic group, for example a group C₁₋₆ alkyl represents a linear or branched carbon-based chain of 1 to 6 carbon atoms, for example a methyl, ethyl, n-propyl, isopropyl, n-butyl, isobutyl, tert-butyl, pentyl or hexyl,
cycloalkyl: an optionally branched, cyclic saturated carbon-based chain containing from 3 to 7 carbon atoms. By way of example, a group C₃₋₇ cycloalkyl represents a hydrocarbon-based chain of 3 to 7 carbon atoms, for example a cyclopropyl, cyclobutyl, cyclopentyl, cyclohexyl or
cycloheptyl,
alkyloxy: a group —O-alkyl,
alkylthio: a group —S-alkyl,
fluoroalkyl: an alkyl group in which one or more hydrogen atoms have been replaced with a fluorine,
fluoroalkyloxy: an alkyloxy group in which one or more hydrogen atoms have been replaced with a fluorine atom.

According to the present invention, among the compounds of formula (I) as defined above, the ones that are particularly preferred are those that have one or a combination of the following characteristics:
R₁ represents a methyl, ethyl or isopropyl group,
R₂ represents a chlorine or bromine atom or a methyl, ethyl, isopropyl or tert-butyl group,
R₃ represents a hydrogen atom,
n=m=2,
A represents either an oxygen atom or a carbon atom substituted with a group —OH,
R₅ represents a group chosen from:
   an unsubstituted phenyl group or a phenyl group substituted with one, two or three identical or different substituents chosen from fluorine, chlorine and bromine atoms and methyl, ethyl, n-butyl, trifluoromethyl, hydroxymethyl, di- and tri-fluoromethoxy, methoxy, phenoxy and benzyloxy groups,
   a group C₂₋₁₂ alkyl, optionally substituted with one or more hydroxyl groups or fluorine atoms,
   a sec-butyl, n-propyl, n-butyl, n-pentyl, 2,2-dimethylpropyl, n-hexyl, n-heptyl, n-octyl or n-nonyl group, an n-butyl group substituted in position 4 with three fluorine atoms, an n-propyl group substituted in position 3 with three fluorine atoms, an n-butyl group substituted in position 4 with a hydroxyl group, or an n-propyl group substituted in position 3 with a hydroxyl group,
   a group —CH₂-cyclopropyl, —CH₂-cyclohexyl, cyclopentyl, cyclohexyl or cycloheptyl,
   a group —(CH₂)ₙ-aryl in which n is equal to 1 or 2 and the aryl group may be optionally substituted with a group Rₐ, preferably in the meta or para position, with a methyl, trifluoromethyl or methoxy group or a fluorine atom.

According to the present invention, among the compounds of formula (I) as defined above, the ones that are more particularly preferred are those that have one or a combination of the following characteristics, when they are not mutually exclusive:
R₁═R₂=iPr, R₃═H,
R₁═R₂=Et, R₃═H,
n=m=2,
A represents either an oxygen atom or a carbon atom substituted with a group —OH,
R₅ represents an unsubstituted phenyl group or a phenyl group substituted, in the meta or para position, with a chlorine or fluorine atom or with a methyl or methoxy group.

The compounds below, and the pharmaceutically acceptable salts, solvates and hydrates thereof and the conformers or rotamers thereof, are particularly preferred:
2-(8-acetyl-2,4-dioxo-1-p-tolyl-1,3,8-triazaspiro[4.5]dec-3-yl)-N-(2,6-diisopropylphenyl)acetamide,
3-[(2,6-diisopropylphenylcarbamoyl)methyl]-2,4-dioxo-1-p-tolyl-1,3,8-triazaspiro[4.5]decane-8-carboxylic acid tert-butyl ester,
2-(8-benzoyl-2,4-dioxo-1-p-tolyl-1,3,8-triazaspiro[4.5]dec-3-yl)-N-(2,6-diisopropylphenyl)acetamide,
2-(8-benzyl-2,4-dioxo-1-p-tolyl-1,3,8-triazaspiro[4.5]dec-3-yl)-N-(2,6-diisopropylphenyl)acetamide,
N-(2,6-diisopropylphenyl)-2-(8-methyl-2,4-dioxo-1-p-tolyl-1,3,8-triazaspiro[4.5]dec-3-yl)acetamide,
N-(2,6-diisopropylphenyl)-2-(8-hydroxy-2,4-dioxo-1-p-tolyl-1,3-diazaspiro[4.5]dec-3-yl)acetamide,
N-(2,6-diisopropylphenyl)-2-(2,4-dioxo-1-p-tolyl-8-thia-1,3-diazaspiro[4.5]dec-3-yl)acetamide,
2-(8,8-difluoro-2,4-dioxo-1-p-tolyl-1,3-diazaspiro[4.5]dec-3-yl)-N-(2,6-diisopropylphenyl)acetamide,
N-(2,6-diisopropylphenyl)-2-(2,4-dioxo-1-p-tolyl-8-oxa-1,3-diazaspiro[4.5]dec-3-yl)acetamide,
N-(2,6-diisopropylphenyl)-2-(2,4,8,8-tetraoxo-1-p-tolyl-8lambda*6*-thia-1,3-diazaspiro[4.5]dec-3-yl)acetamide,
N-(2,6-diethylphenyl)-2-(2,4-dioxo-1-p-tolyl-8-oxa-1,3-diazaspiro[4.5]dec-3-yl)acetamide,
N-(2,6-diisopropylphenyl)-2-[1-(4-methoxyphenyl)-2,4-dioxo-8-oxa-1,3-diazaspiro[4.5]dec-3-yl]acetamide,
N-(2,6-diisopropylphenyl)-2-[1-(3-methoxyphenyl)-2,4-dioxo-8-oxa-1,3-diazaspiro[4.5]dec-3-yl]acetamide,
N-(2,6-diisopropylphenyl)-2-[1-(4-methylsulfanylphenyl)-2,4-dioxo-8-oxa-1,3-diazaspiro[4.5]dec-3-yl]-acetamide.

A subject of the invention is also a process for preparing the compounds of general formula (I).

In accordance with the invention, the compounds of formula (I) may be prepared according to the general process described in Scheme 1 below.

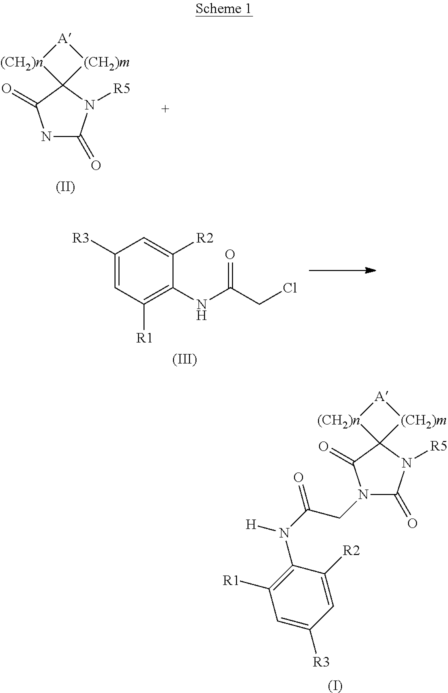

The compounds of formula (I) in which R₁, R₂, R₃, A, n, m and R₅ are as defined above may be prepared by reacting the dioxo-imidazolidines of formula (II) with the chloroacetamides of formula (III), in the presence of a base, according to Scheme 1 and by analogy, for example, with the reactions described by Dunbar, B. et al., Pharmazie 2002, 57 (7), 438, Pinza, M. et al., J. Med. Chem. 1993, 36 (26), 4214, Coudert, P. et al., Pharm. Acta Helv. 1991, 66 (5-6), 155 or Usifoh, C. O.; Arch. Pharm. 2001, 334 (11), 366. The group A′ represents either the group A defined above or a precursor of A converted into A via a method known to those skilled in the art.

### Synthesis of the Intermediates (II) and (III)

The dioxo-imidazolidines of general formula (II) in which A, n and m are as defined above for the compounds of formula (I) may be prepared according to Scheme 2 below: in particular, the group A′ represents either the group A defined above or a precursor of A converted into A via a method known to those skilled in the art.

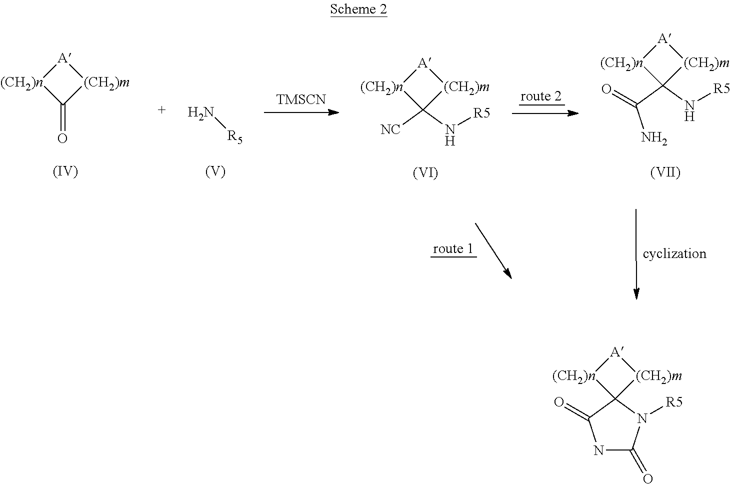

The nitrile compounds of formula (VI) are obtained from the ketones of formula (IV) reacted with the amines of formula (V) in the presence of trimethylsilyl cyanide, in accordance, for example, with the conditions described in Matsumoto K. et al., Helv. Chim. Acta 2005, 88 (7), 1734-1753 or Nieto M. J. et al., J. Comb. Chem. 2005, 7 (2), 258-263.

The ketones (IV) and the anilines (V) are commercial compounds or are prepared according to techniques that are well known to those skilled in the art.

Scheme 2 Route 1 Method 1

The dioxo-imidazolidine intermediates of formula (II) may be prepared by reacting the nitrile derivatives (VI) with potassium isocyanate, followed by work-up in acidic medium according, for example, to the conditions described in patent DE 1 032 258.

Scheme 2 Route 1 Method 2

The dioxo-imidazolidine intermediates of formula (II) may also be prepared by reacting the nitrile derivatives (VI) with chlorosulfonyl isocyanate, followed by work-up in acidic medium according, for example, to Feldman Paul L. et al., J. Org. Chem. 1990, 4207 or Goebel Tim et al., J. Med. Chem. 2008, 238.

Scheme 2 Route 2

Hydrolysis of the nitrile function of the compounds of formula (VI) in the presence of acid, for example under the conditions described in Beths R. L. et al., J. Chem. Soc., 1927, 1310, makes it possible to obtain the primary amides of formula (VII). Cyclization in the presence of a suitable aryl isocyanate as described in Papadopoulos, E. P.; J. Org. Chem. 1977, 42, 3925 makes it possible to obtain the dioxoimidazolidines of formula (II).

The chloroacetamides of general formula (III) may be prepared by reaction between the anilines of formula (VIII) and chloroacetyl chloride in the presence of a base, for example as described in Davion, Y. et al., Heterocycles 2004, 63 (5), 1093 or in Juaristi, E. et al., J. Org. Chem. 1999, 64 (8), 2914, as illustrated in Scheme 3 below in which R₁, R₂ and R₃ are as defined for the compounds of formula (I):

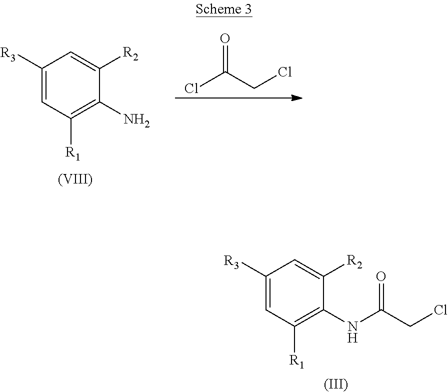

The functional groups that may be present in the reaction intermediates used in the process may be protected, either permanently or temporarily, with protecting groups that ensure an unequivocal synthesis of the expected compounds. The protection and deprotection reactions are performed according to techniques that are well known to those skilled in the art. The term “temporary protecting group for amines, alcohols or carboxylic acids” means protecting groups such as those described in “Protective Groups in Organic Chemistry”, published by McOmie J. W. F., Plenum Press, 1973, in “Protective Groups in Organic Synthesis”, 2nd edition, Greene T. W. and Wuts P. G. M., published by John Wiley & Sons, 1991, and in “Protecting Groups”, Kocienski P. J., 1994, Georg Thieme Verlag.

The compounds (I) according to the invention, and also the pharmaceutically acceptable salts, solvates and/or hydrates thereof, have inhibitory properties on the enzyme SOAT-1. This inhibitory activity on the enzyme SOAT-1 is measured according to a HepG2 primary enzymatic test, as described in Example 15. The preferred compounds of the present invention have a concentration that enables inhibition of 50% of the response of the enzyme (IC₅₀) of less than or equal to 1000 nM, preferentially less than or equal to 300 nM and advantageously less than or equal to 50 nM.

A subject of the present invention is also, as medicaments, the compounds of formula (I) as described above, and also the pharmaceutically acceptable salts and pharmaceutically acceptable solvates and/or hydrates thereof.

A subject of the present invention is the use of at least one compound of formula (I), or pharmaceutically acceptable salts or solvates and/or hydrates thereof, for the manufacture of a medicament for preventing and/or treating sebaceous gland disorders such as hyperseborrhoea, acne, seborrhoeic dermatitis or atopic dermatitis, ocular pathologies such as blepharitis or meibomitis (disorder of the Meibomius gland) or pathologies such as hypercholesterolaemia, arteriosclerosis or Alzheimer's disease. The compounds according to the invention are particularly suitable for the manufacture of a pharmaceutical composition for treating acne. The compounds according to the invention are thus suitable for use in the pathologies listed above.

A subject of the present invention is also a pharmaceutical or cosmetic composition comprising, in a physiologically acceptable support, at least one compound of formula (I) as defined above, or a pharmaceutically acceptable salt or solvate and/or hydrate thereof. The compositions according to the invention thus comprise a physiologically acceptable support or at least one physiologically or pharmaceutically acceptable excipient, chosen according to the desired cosmetic or pharmaceutical form and the chosen mode of administration.

The term “physiologically acceptable support or medium” means a support that is compatible with the skin, mucous membranes and/or the integuments.

The administration of the composition according to the invention may be performed via the enteral, parenteral, rectal, topical or ocular route. Preferably, the pharmaceutical composition is conditioned in a form that is suitable for topical application.

Via the enteral route, the composition, more particularly the pharmaceutical composition, may be in the form of tablets, gel capsules, coated tablets, syrups, suspensions, solutions, powders, granules, emulsions, microspheres or nanospheres or lipid or polymer vesicles allowing controlled release. Via the parenteral route, the composition may be in the form of solutions or suspensions for perfusion or for injection.

The compositions according to the invention contain a compound according to the invention, in an amount sufficient to obtain the desired therapeutic, prophylactic or cosmetic effect. The compounds according to the invention are generally administered at a daily dose of about 0.001 mg/kg to 100 mg/kg of body weight, in 1 to 3 dosage intakes. The compounds are used systemically at a concentration generally of between 0.001% and 10% by weight and preferably between 0.01% and 5% by weight relative to the weight of the composition.

Via the topical route, the pharmaceutical composition according to the invention is more particularly intended for treating the skin and mucous membranes and may be in the form of ointments, creams, milks, pomades, powders, impregnated pads, syndets, solutions, gels, sprays, mousses, suspensions, lotions, sticks, shampoos or washing bases. It may also be in the form of suspensions of microspheres or nanospheres or lipid or polymer vesicles or polymer patches and hydrogels allowing controlled release. This topical composition may be in anhydrous form, in aqueous form or in the form of an emulsion.

The compounds are used topically at a concentration generally of between 0.001% and 10% by weight and preferably between 0.01% and 5% by weight relative to the total weight of the composition.

The compounds of formula (I) according to the invention and the pharmaceutically acceptable salts or solvates and/or hydrates thereof also find an application in the cosmetics field, in particular in body and hair hygiene and more particularly for combating or preventing greasy skin or hair or a greasy scalp.

A subject of the invention is thus also the cosmetic use of a composition comprising, in a physiologically acceptable support, at least one of the compounds of formula (I), optionally in the form of a pharmaceutically acceptable salt or solvate and/or hydrate, for body or hair hygiene.

The cosmetic composition according to the invention containing, in a cosmetically acceptable support, at least one compound of formula (I) or a pharmaceutically acceptable salt or solvate and/or hydrate thereof may especially be in the form of a cream, a milk, a lotion, a gel, an ointment, a pomade, a suspension of microspheres or nanospheres or lipid or polymer vesicles, impregnated pads, solutions, sprays, mousses, sticks, soaps, shampoos or washing bases.

The pharmaceutical and cosmetic compositions as described previously may also contain inert or even pharmacodynamically active additives as regards the pharmaceutical compositions, or combinations of these additives, and especially:
wetting agents;
flavour enhancers;
preserving agents such as para-hydroxybenzoic acid esters;
stabilizers;
humidity regulators;
pH regulators;
osmotic pressure modifiers;
emulsifiers;
UV-A and UV-B screening agents;
antioxidants, such as α-tocopherol, butylhydroxyanisole or butylhydroxytoluene, superoxide dismutase, ubiquinol or certain metal-chelating agents;
emollients;
moisturizers, for instance glycerol, PEG-400, thiamorpholinone and derivatives thereof, or urea;
carotenoids and especially β-carotene;
α-hydroxy acids and α-keto acids or derivatives thereof, such as lactic acid, malic acid, citric acid, glycolic acid, mandelic acid, tartaric acid, glyceric acid or ascorbic acid, and also salts, amides or esters thereof, or β-hydroxy acids or derivatives thereof, such as salicylic acid and salts, amides or esters thereof.

Needless to say, a person skilled in the art will take care to select the optional compound(s) to be added to these compositions such that the advantageous properties intrinsically associated with the present invention are not, or are not substantially, adversely affected by the envisaged addition. Moreover, in general, the same preferences as those indicated previously for the compounds of formula (I) apply mutatis mutandis to the medicaments and cosmetic and pharmaceutical compositions and to the use using the compounds of the invention.

Several examples of preparation of active compounds of formula (I) according to the invention, and the results of the biological activity of such compounds, are given hereinbelow as illustrations and with no limiting nature.

### PROCEDURES

### Example 1

### 2-(8-acetyl-2,4-dioxo-1-p-tolyl-1,3,8-triazaspiro[4.5]dec-3-yl)-N-(2,6-diisopropylphenyl)acetamide

### Step 1.14-cyano-4-p-tolylaminopiperidine-1-carboxylic acid tert-butyl ester

2.7 g (25.2 mmol, 1 eq.) of p-toluidine (starting material 1) are added to a solution of 5 g (25.1 mmol, 1 eq.) of 1-tert-butyloxycarbonyl-4-piperidone (starting material 2) in 30 ml of acetic acid at 0° C. The solution is stirred for 15 minutes, and 3.4 ml (25.5 mmol, 1 eq.) of trimethylsilyl cyanide are added. The reaction medium is stirred for 4 hours at room temperature. It is then poured slowly into an ice-cold ammonium hydroxide solution and extracted with ethyl acetate. The organic phases are combined and washed with water. They are dried over sodium sulfate. The residue is precipitated from a mixture of dichloromethane and heptane. It is filtered off and dried. 4-Cyano-4-p-tolylaminopiperidine-1-carboxylic acid tert-butyl ester is obtained in the form of a white solid.

Melting point=107-109° C.

### Step 1.2 1-p-tolyl-1,3,8-triazaspiro[4.5]decane-2,4-dione

Preparation According to Scheme 2, route 1, method 1

1.23 g (18.9 mmol, 1.5 eq.) of potassium cyanate are added at 35° C. to a solution of 4 g (12.7 mmol, 1 eq.) of 4-cyano-4-p-tolylaminopiperidine-1-carboxylic acid tert-butyl ester in 20 ml of glacial acetic acid. The reaction medium is stirred at 60° C. for 2 hours. 4 ml of hydrochloric acid and then 2.7 ml of water are added. The medium is heated at 90° C. for 1 hour and then at room temperature for 1 hour. It is then poured into 100 ml of water. The aqueous phase is evaporated to 75% and the precipitate is triturated in a small volume of dichloromethane. It is filtered off and dried. 1-p-Tolyl-1,3,8-triazaspiro[4.5]decane-2,4-dione is obtained in the form of a white solid. Melting point=128-130° C.

### Step 1.3 8-acetyl-1-p-tolyl-1,3,8-triazaspiro[4.5]decane-2,4-dione

75 μl (1.05 mmol, 1 eq.) of acetyl chloride are added to a solution of 270 mg (1.04 mmol, 1 eq.) of 1-p-tolyl-1,3,8-triazaspiro[4.5]decane-2,4-dione in 20 ml of dichloromethane. The medium is stirred at room temperature for 24 hours. It is evaporated and the residue is chromatographed on silica gel (50/50 heptane/ethyl acetate and then ethyl acetate). 8-Acetyl-1-p-tolyl-1,3,8-triazaspiro[4.5]decane-2,4-dione is obtained in the form of a white solid.

Melting point=295-297° C.

### Step 1.4 2-(8-acetyl-2,4-dioxo-1-p-tolyl-1,3,8-triazaspiro[4.5]dec-3-yl)-N-(2,6-diisopropylphenyl)acetamide

Preparation According to Scheme 1

37 mg (0.26 mmol, 1 eq.) of potassium carbonate are added to a solution of 80 mg (0.26 mmol, 1 eq.) of 8-acetyl-1-p-tolyl-1,3,8-triazaspiro[4.5]decane-2,4-dione and 68 mg (0.26 mmol, 1 eq.) of 2-chloro-N-(2,6-diisopropylphenyl)acetamide in 15 ml of dimethylformamide. The reaction medium is stirred at room temperature for 24 hours. It is then poured into water and extracted with ethyl acetate. The organic phases are combined and washed with water. They are dried over sodium sulfate. The solvents are evaporated off. The residue is precipitated from a mixture of dichloromethane and heptane. 2-(8-Acetyl-2,4-dioxo-1-p-tolyl-1,3,8-triazaspiro[4.5]dec-3-yl)-N-(2,6-diisopropylphenyl)acetamide is obtained in the form of a white solid.

Melting point=136-138° C.

NMR (CDCl₃): 1.20 (s, 6H); 1.22 (s, 6H); 1.74-1.98 (m, 4H); 2.06 (s, 3H); 2.41 (s, 3H); 3.06-3.37 (m, 2H); 3.31-3.92 (m, 4H); 4.49 (s, 2H); 4.55-4.59 (m, 1H); 7.03-7.06 (m, 2H); 7.13-7.40 (m, 5H)

### Preparation of the intermediate 2-chloro-N-(2,6-diisopropylphenyl)acetamide

Synthesis according to Scheme 3

222 mL (1.59 mol) of triethylamine are added to a solution of 300 mL (1.59 mol) of 2,6-diisopropylphenylamine (Starting material 3) in 1 litre of dichloromethane. The reaction mixture is cooled to 0° C., and 127 mL (1.59 mol) of chloroacetyl chloride are then added dropwise. Once the addition is complete, the ice bath is removed and the medium is stirred for 20 minutes. It is then poured into water and extracted with dichloromethane. The organic phases are combined and washed with water. They are dried over sodium sulfate. The solvents are evaporated off. The residue is filtered through silica gel (eluent: dichloromethane). The filtrate is evaporated and then triturated in heptane. 2-Chloro-N-(2,6-diisopropylphenyl)acetamide is obtained in the form of a white solid.

Melting point=146-148° C.

### Example 2

### 3-[(2,6-diisopropylphenylcarbamoyl)methyl]-2,4-dioxo-1-p-tolyl-1,3,8-triazaspiro[4.5]decane-8-carboxylic acid tert-butyl ester

### Step 2.1 2,4-dioxo-1-p-tolyl-1,3,8-triazaspiro[4.5]decane-8-carboxylic acid tert-butyl ester

337 mg (1.54 mmol, 1 eq.) of di-tert-butyl dicarbonate and 220 μl (1.57 mmol, 1.02 eq.) of triethylamine are added to a solution of 400 mg (1.54 mmol, 1 eq.) of 1-p-tolyl-1,3,8-triazaspiro[4.5]decane-2,4-dione in 20 ml of dichloromethane. The reaction medium is stirred for 4 hours at room temperature. It is then evaporated, triturated in heptane and filtered. 2,4-Dioxo-1-p-tolyl-1,3,8-triazaspiro[4.5]decane-8-carboxylic acid tert-butyl ester is obtained in the form of a white solid.

Melting point=223-225° C.

### Step 2.2 3-[(2,6-diisopropylphenylcarbamoyl)methyl]-2,4-dioxo-1-p-tolyl-1,3,8-triazaspiro[4.5]decane-8-carboxylic acid tert-butyl ester

Preparation According to Scheme 1

This compound is prepared according to the procedure described in step 1.4 above, starting with 440 mg of 2,4-dioxo-1-p-tolyl-1,3,8-triazaspiro[4.5]decane-8-carboxylic acid tert-butyl ester. Melting point=227-229° C.

NMR (CDCl₃): 1.20 (s, 6H); 1.22 (s, 6H); 1.41 (s, 9H); 1.70-2.0 (m, 4H); 2.41 (s, 3H); 3.07-3.10 (m, 2H); 3.45-4.20 (m, 4H); 4.48 (s, 2H); 7.05-7.07 (d, 2H); 7.19-7.32 (m, 5H)

### Example 3

### 2-(8-benzoyl-2,4-dioxo-1-p-tolyl-1,3,8-triazaspiro[4.5]dec-3-yl)-N-(2,6-diisopropylphenyl)acetamide

### Step 3.1 8-benzoyl-1-p-tolyl-1,3,8-triazaspiro[4.5]decane-2,4-dione

90 μl (0.77 mmol, 1 eq.) of benzoyl chloride are added to a solution of 200 mg (0.77 mmol, 1 eq.) of 1-p-tolyl-1,3,8-triazaspiro[4.5]decane-2,4-dione in 20 ml of dichloromethane. The medium is stirred at room temperature for 4 hours. It is evaporated and the residue is precipitated from dichloromethane and heptane. 8-Benzoyl-1-p-tolyl-1,3,8-triazaspiro[4.5]decane-2,4-dione is obtained in the form of a white solid.

Melting point=242-244° C.

### Step 3.2 2-(8-benzoyl-2,4-dioxo-1-p-tolyl-1,3,8-triazaspiro[4.5]dec-3-yl)-N-(2,6-diisopropylphenyl)acetamide

Preparation According to Scheme 1

This compound is prepared according to the procedure described in step 1.4 above, starting with 150 mg of 1-(6-methylpyridin-3-yl)-1,3-diazaspiro[4.5]decane-2,4-dione.

Melting point=262-264° C.

NMR (CDCl₃): 0.90 (s, 6H); 091 (s, 6H); 1.70-2.10 (m, 4H); 2.43 (s, 3H); 3.07-3.10 (m, 2H); 3.50-3.90 (m, 4H); 4.48 (s, 2H); 4.60-4.80 (m, 1H); 7.04-7.07 (m, 2H); 7.13-7.41 (m, 10H)

### Example 4

### 2-(8-benzyl-2,4-dioxo-1-p-tolyl-1,3,8-triazaspiro[4.5]dec-3-yl)-N-(2,6-diisopropylphenyl)acetamide

### Step 4.1 8-benzyl-1-p-tolyl-1,3,8-triazaspiro[4.5]decane-2,4-dione

90 μl (0.77 mmol, 1 eq.) of benzyl bromide are added to a solution of 200 mg (0.77 mmol, 1 eq.) of 1-p-tolyl-1,3,8-triazaspiro[4.5]decane-2,4-dione in 20 ml of dichloromethane. The medium is stirred at room temperature for 24 hours. It is evaporated and the residue is chromatographed on silica gel (ethyl acetate). 8-Benzyl-1-p-tolyl-1,3,8-triazaspiro[4.5]decane-2,4-dione is obtained in the form of a white solid.

Melting point=291-293° C.

### Step 4.2 2-(8-benzyl-2,4-dioxo-1-p-tolyl-1,3,8-triazaspiro[4.5]dec-3-yl)-N-(2,6-diisopropylphenyl)acetamide

Preparation According to Scheme 1

This compound is prepared according to the procedure described in step 1.4 above, starting with 60 mg of 8-benzyl-1-p-tolyl-1,3,8-triazaspiro[4.5]decane-2,4-dione.

Melting point=206-208° C.

NMR (CDCl₃): 1.20 (s, 6H); 1.22 (s, 6H); 1.94-1.97 (m, 4H); 2.41 (s, 3H); 2.77-2.83 (m, 4H); 3.06-3.08 (m, 2H); 3.53-3.55 (m, 2H); 4.47 (s, 2H); 7.05-7.07 (m, 2H); 7.18-7.31 (m, 6H)

### Example 5

### N-(2,6-diisopropylphenyl)-2-(8-methyl-2,4-dioxo-1-p-tolyl-1,3,8-triazaspiro[4.5]dec-3-yl)acetamide

### Step 5.1 8-methyl-1-p-tolyl-1,3,8-triazaspiro[4.5]decane-2,4-dione

1 ml (13.3 mmol, 6.9 eq.) of 37% formaldehyde and 600 μl (13.86 mmol, 7.2 eq.) of formic acid are added to 500 mg (1.93 mmol, 1 eq.) of 1-p-tolyl-1,3,8-triazaspiro[4.5]decane-2,4-dione. The medium is stirred at 80° C. for 4 hours. It is then poured into water and basified with sodium hydroxide to basic pH and extracted with ethyl acetate. The organic phases are combined and washed with water. They are dried over sodium sulfate. The solvents are evaporated off. The residue is precipitated from dichloromethane and heptane. 8-Methyl-1-p-tolyl-1,3,8-triazaspiro[4.5]decane-2,4-dione is obtained in the form of a white solid.

Melting point=172-174° C.

### Step 5.2 N-(2,6-diisopropylphenyl)-2-(8-methyl-2,4-dioxo-1-p-tolyl-1,3,8-triazaspiro[4.5]dec-3-yl)acetamide

Preparation According to Scheme 1

This compound is prepared according to the procedure described in step 1.4 above, starting with 140 mg of 8-methyl-1-p-tolyl-1,3,8-triazaspiro[4.5]decane-2,4-dione.

Melting point=232-234° C.

NMR (CDCl₃): 1.20 (s, 6H); 1.22 (s, 6H); 1.87-2.02 (m, 4H); 2.32 (s, 3H); 2.36 (s, 3H); 2.70-2.81 (m, 4H); 3.07-3.10 (m, 2H); 4.47 (s, 2H); 7.05-7.09 (m, 2H); 7.18-7.33 (m, 6H)

### Example 6

### N-(2,6-Diisopropylphenyl)-2-(8-hydroxy-2,4-dioxo-1-p-tolyl-1,3-diazaspiro[4.5]dec-3-yl)acetamide

### Step 6.1 4-(tert-Butyldimethylsilanyloxy)-1-p-tolylaminocyclohexanecarbonitrile

This compound is prepared according to the procedure described in step 1.1 above, starting with 1.2 g of p-toluidine and 3 g of 4-(tert-butyldimethylsiloxy)cyclohexanone.

The product obtained is a yellow oil.

### Step 6.2 Carbamic acid 2,4-dioxo-1-p-tolyl-1,3-diazaspiro[4.5]dec-8-yl ester

Preparation According to Scheme 2, route 1, method 1

This compound is prepared according to the procedure described in step 1.2 above, starting with 3.6 g of 4-(tert-butyldimethylsilanyloxy)-1-p-tolylaminocyclohexanecarbonitrile.

Melting point=189-191° C.

### Step 6.3 Carbamic acid 3-[(2,6-diisopropylphenylcarbamoyl)methyl]-2,4-dioxo-1-p-tolyl-1,3-diazaspiro[4.5]dec-8-yl ester

Preparation According to Scheme 1

This compound is prepared according to the procedure described in step 1.4 above, starting with 800 mg of carbamic acid 2,4-dioxo-1-p-tolyl-1,3-diazaspiro[4.5]dec-8-yl ester.

Melting point=245-247° C.

### Step 6.4 N-(2,6-Diisopropylphenyl)-2-(8-hydroxy-2,4-dioxo-1-p-tolyl-1,3-diazaspiro[4.5]dec-3-yl)acetamide

200 μl (1.12 mmol, 2 eq.) of sodium methoxide dissolved in methanol (5.4 M) are added to a solution at −10° C. of 300 mg (0.56 mmol, 1 eq.) of carbamic acid 3-[(2,6-diisopropylphenylcarbamoyl)methyl]-2,4-dioxo-1-p-tolyl-1,3-diazaspiro[4.5]dec-8-yl ester in 10 ml of methanol. The reaction medium is allowed to warm to room temperature and is then heated at 50° C. for 4 hours. The methanol is then evaporated off and the residue is taken up in ethyl acetate and washed with water. The organic phases are combined and washed with water. They are dried over sodium sulfate. The solvents are evaporated off. The residue is chromatographed on silica gel (40/60 ethyl acetate/heptane). N-(2,6-Diisopropylphenyl)-2-(8-hydroxy-2,4-dioxo-1-p-tolyl-1,3-diazaspiro[4.5]dec-3-yl)acetamide is obtained in the form of a white solid.

Melting point=142-144° C.

NMR (CDCl₃): 1.21 (s, 6H); 1.23 (s, 6H); 1.72-1.86 (m, 4H); 2.07-2.2 (m, 2H); 2.33-2.39 (m, 2H); 2.41 (s, 3H); 3.06-3.13 (m, 2H); 4.06-4.10 (m, 1H); 4.48 (s, 2H); 7.10-7.42 (m, 8H)

### Example 10

### N-(2,6-Diisopropylphenyl)-2-(2,4,8,8-tetraoxo-1-p-tolyl-8lambda*6*-thia-1,3-diazaspiro[4.5]dec-3-yl)acetamide

0.275 g (0.445 mmol; 2.2 eq.) of oxone is added to a solution of 0.1 g (0.2 mmol; 1 eq.) of N-(2,6-diisopropylphenyl)-2-(2,4-dioxo-1-p-tolyl-8-thia-1,3-diazaspiro[4.5]dec-3-yl)acetamide (prepared according to Example 1) in 3 ml of methanol and 1 ml of demineralized water. The reaction medium is stirred at room temperature for 24 hours. Water is added, followed by dichloromethane. The organic phase is washed several times with water. It is then dried over magnesium sulfate, filtered and then concentrated to dryness. The white solid is purified on silica gel and eluted with a gradient of from 100% dichloromethane to a 95/5 mixture of dichloromethane/methanol. The expected product is obtained in the form of a white solid.

Melting point=294° C.

NMR (DMSO): 1.07-1.14 (12H, m); 2.13-2.19 (2H, m); 2.37 (3H, s); 2.47-2.50 (2H, m); 3.01-3.09 (2H, m); 3.19-3.22 (2H, m); 3.56-3.63 (2H, m); 4.34 (2H, s); 7.15-7.17 (2H, m); 7.20 (2H, d, J=8.15 Hz); 7.24-7.28 (1H, m); 7.34 (2H, d, J=8.15 Hz); 9.57 (1H, s)

### Example 12

### N-(2,6-Diisopropylphenyl)-2-[1-(4-methoxyphenyl)-2,4-dioxo-8-oxa-1,3-diazaspiro[4.5]dec-3-yl]acetamide

### Step 12.1 4-(4-Methoxyphenylamino)tetrahydropyran-4-carboxylic acid amide

Preparation According to Scheme 2, Route 2

2.7 g (11.6 mmol) of 4-(4-methoxyphenylamino)tetrahydropyran-4-carbonitrile (prepared according to Step 1.1 above) are dissolved in 10 ml of concentrated sulfuric acid. The reaction medium is stirred at room temperature overnight. It is poured gently into ice-cold water and the pH is brought to 12 with aqueous 10N sodium hydroxide. The precipitate formed is filtered off and dried. 4-(4-Methoxyphenylamino)tetrahydropyran-4-carboxylic acid amide is obtained in the form of a white solid.

Melting point=141-143° C.

### Step 12.2 1-(4-Methoxyphenyl)-1,3-diazaspiro[4.5]decane-2,4-dione

300 μl (1.46 mmol, 1.2 eq.) of 2,6-diisopropylphenyl isocyanate are added to a solution of 300 mg (1.20 mmol, 1 eq.) of 4-(4-methoxyphenylamino)tetrahydropyran-4-carboxylic acid amide in ml of toluene. The reaction medium is stirred at 180° C. for 2 hours under microwave irradiation. The toluene is evaporated off and the residue is purified on silica gel (heptane and then an increasing percentage of ethyl acetate). 1-(4-Methoxyphenyl)-1,3-diazaspiro[4.5]decane-2,4-dione is obtained in the form of a white solid.

Melting point=203-205° C.

### Step 12.3 N-(2,6-Diisopropylphenyl)-2-[1-(4-methoxyphenyl)-2,4-dioxo-8-oxa-1,3-diazaspiro[4.5]dec-3-yl]acetamide

This compound is prepared in the form of a white solid according to the procedure described in Step 1.4 above, starting with 150 mg of 1-(4-methoxyphenyl)-1,3-diazaspiro[4.5]decane-2,4-dione.

Melting point=232-234° C.

NMR (DMSO): 1.08 (s, 6H); 1.11 (s, 6H); 1.64-1.72 (m, 2H); 1.90-1.93 (m, 2H); 3.03-3.10 (m, 2H); 3.71-3.75 (m, 2H); 3.80 (s, 3H); 3.89-3.94 (m, 2H); 4.30 (s, 2H); 7.03-7.06 (d, 2H); 7.14-7.19 (m, 4H); 7.24-7.27 (m, 1H); 9.53 (s, 1H)

### Example 13

### N-(2,6-Diisopropylphenyl)-2-[1-(4-methylsulfanylphenyl)-2,4-dioxo-8-oxa-1,3-diazaspiro[4.5]dec-3-yl]acetamide

### Step 13.1 1-(4-Methylsulfanylphenyl)-8-oxa-1,3-diazaspiro[4.5]decane-2,4-dione

Preparation According to Scheme 2 route 1, method 2

0.227 g (1.6 mmol, 2 eq.) of chlorosulfonyl isocyanate is added dropwise to a solution of 0.2 g (0.8 mmol) of 4-(4-methylsulfanylphenylamino)tetrahydropyran-4-carbonitrile (prepared according to Step 1.2 above) in 10 ml of chloroform. The reaction mixture turns pale green and cloudy and the slightly exothermic reaction (Tmax ˜40° C.) is stirred for two hours at room temperature. 20 ml of 1N HCl are added and the reaction medium is refluxed for 16 hours. On reaching 80° C., the medium becomes pale orange and clear. A precipitate is detected after 72 hours at room temperature. After filtering off and washing with 2×20 ml of water, a white solid is obtained, corresponding to the expected product. This product is used in the next step of the synthesis without further purification.

### Step 13.2 N-(2,6-Diisopropylphenyl)-2-[1-(4-methylsulfanylphenyl)-2,4-dioxo-8-oxa-1,3-diazaspiro[4.5]dec-3-yl]acetamide

This compound is prepared in the form of fine needles according to the procedure described in Step 1.4 above, starting with 0.1 g of 1-(4-methylsulfanylphenyl)-8-oxa-1,3-diazaspiro[4.5]decane-2,4-dione. Melting point 240-240° C.

NMR (DMSO): 1.08 (s, 6H); 1.12 (s, 6H); 1.70-1.78 (m, 2H); 1.92-1.96 (m, 2H); 3.03-3.10 (m, 2H); 3.72-3.76 (m, 2H); 3.78 (s, 3H); 3.89-3.95 (m, 2H); 4.31 (s, 2H); 6.83-6.86 (m, 2H); 7.05-7.08 (dd, 1H, J=2.04 Hz, J′=5.96 Hz); 7.14-7.16 (d, 2H, J=7.6 Hz); 7.24-7.27 (m, 1H); 7.39-7.44 (m, 1H); 9.54 (s, 1H)

### Examples 7, 8, 9, 11 and 14

Examples 7, 8, 9, 11 and 14 are described in Table 1 below. The compounds are synthesized according to the above procedures, replacing the starting materials 1, 2 and 3 mentioned in Examples 1, 2, 3, 4, 5, 6, 10, 12 and 13 with the products mentioned in Table 1.

TABLE 1

| | | | | | | | NMR 1H 400 MHz |
|---|---|---|---|---|---|---|---|
| | | | | | | | (s = singlet, |
| | | | | | | | d = doublet, t = |
| | | | | | | | triplet, q = quartet, |
| | | | | | Synthetic | Melting | m = multiplet, J = |
| Example | | Starting | Starting | Starting | route | point | coupling constant |
| # | IUPAC name | material 1 | material 2 | material 3 | Scheme 2 | (° C.) | in Hz) |
| 1 | 2-(8-acetyl-2,4- | p-toluidine | 1-boc-4-piper- | 2,6-diiso- | route 1 | 136-138 | (CDCl₃): 1.20 (s, 6H); |
| | dioxo-1-p-tolyl- | | idone | propyl-phenyl- | method 1 | | 1.22 (s, 6H); 1.74-1.98 |
| | 1,3,8-triazaspiro- | | | amine | | | (m, 4H); 2.06 (s, 3H); |
| | [4.5]dec-3-yl)-N- | | | | | | 2.41 (s, 3H); 3.06-3.37 |
| | (2,6-diisopropyl- | | | | | | (m, 2H); 3.31-3.92 |
| | phenyl) acetamide. | | | | | | (m, 4H); 4.49 (s, 2H); |
| | | | | | | | 4.55-4.59 (m, 1H); |
| | | | | | | | 7.03-7.06 (m, 2H); |
| | | | | | | | 7.13-7.40 (m, 5H) |
| 2 | 3-[(2,6-diisopropyl- | p-toluidine | 1-boc-4-piper- | 2,6-diiso- | route 1 | 227-229 | (CDCl₃): 1.20 (s, 6H); |
| | phenylcarbamoyl)- | | idone | propyl-phenyl- | method 1 | | 1.22 (s, 6H); 1.41 |
| | methyl]-2,4-dioxo-1-p- | | | amine | | | (s, 9H); 1.70-2.0 |
| | tolyl-1,3,8-triaza- | | | | | | (m, 4H); 2.41 (s, 3H); |
| | spiro[4.5]decane-8- | | | | | | 3.07-3.10 (m, 2H); |
| | carboxylic acid tert- | | | | | | 3.45-4.20 (m, 4H); |
| | butyl ester. | | | | | | 4.48 (s, 2H); 7.05-7.07 |
| | | | | | | | (d, 2H); 7.19-7.32 |
| | | | | | | | (m, 5H) |
| 3 | 2-(8-benzoyl-2,4- | p-toluidine | 1-boc-4-piper- | 2,6-diiso- | route 1 | 262-264 | (CDCl₃): 0.90 (s, 6H); |
| | dioxo-1-p-tolyl- | | idone | propyl-phenyl- | method 1 | | 091 (s, 6H); 1.70-2.10 |
| | 1,3,8-triazaspiro- | | | amine | | | (m, 4H); 2.43 (s, 3H); |
| | [4.5]dec-3-yl)-N- | | | | | | 3.07-3.10 (m, 2H); |
| | (2,6-diisopropyl- | | | | | | 3.50-3.90 (m, 4H); 4.48 |
| | phenyl)-acetamide. | | | | | | (s, 2H); 4.60-4.80 |
| | | | | | | | (m, 1H); 7.04-7.07 |
| | | | | | | | (m, 2H); 7.13-7.41 (m, |
| | | | | | | | 10H) |
| 4 | 2-(8-benzyl-2,4- | p-toluidine | 1-boc-4-piper- | 2,6-diiso- | route 1 | 206-208 | (CDCl₃) : 1.20 (s, 6H); |
| | dioxo-1-p-tolyl- | | idone | propyl-phenyl- | method 1 | | 1.22 (s, 6H); 1.94-1.97 |
| | 1,3,8-triazaspiro- | | | amine | | | (m, 4H); 2.41 (s, 3H); |
| | [4.5]dec-3-yl)-N- | | | | | | 2.77-2.83 (m, 4H); 3.06- |
| | (2,6-diisopropyl- | | | | | | 3.08 (m, 2H); 3.53-3.55 |
| | phenyl)-acetamide. | | | | | | (m, 2H); 4.47 (s, 2H); |
| | | | | | | | 7.05-7.07 (m, 2H); 7.18- |
| | | | | | | | 7.31 (m, 6H) |
| 5 | N-(2,6-diisopropyl- | p-toluidine | 1-boc-4-piper- | 2,6-diiso- | route 1 | 232-234 | (CDCl₃): 1.20 (s, 6H); |
| | phenyl)-2-(8-methyl- | | idone | propyl-phenyl- | method 1 | | 1.22 (s, 6H); 1.87-2.02 |
| | 2,4-dioxo-l-p-tolyl- | | | amine | | | (m, 4H); 2.32 (s, 3H); |
| | 1,3,8-triazaspiro- | | | | | | 2.36 (s, 3H); 2.70-2.81 |
| | [4.5]dec-3-yl) acet- | | | | | | (m, 4H); 3.07-3.10 (m, 2H); |
| | amide. | | | | | | 4.47 (s, 2H); 7.05-7.09 |
| | | | | | | | (m, 2H); 7.18-7.33 (m, 6H) |
| 6 | N-(2,6-diisopropyl- | p-toluidine | 4-(tert-butyl- | 2,6-diiso- | route 1 | 142-144 | (CDCl₃): 1.21 (s, 6H); |
| | phenyl)-2-(8-hydroxy- | | dimethyl-siloxy)- | propyl-phenyl- | method 1 | | 1.23 (s, 6H); 1.72-1.86 |
| | 2,4-dioxo-1-p-tolyl- | | cyclohexanone | amine | | | (m, 4H); 2.07-2.2 (m, 2H); |
| | 1,3-diazaspiro[4.5]dec- | | | | | | 2.33-2.39 (m, 2H); 2.41 |
| | 3-yl)-acetamide. | | | | | | (s, 3H); 3.06-3.13 (m, 2H); |
| | | | | | | | 4.06-4.10 (m, 1H); 4.48 |
| | | | | | | | (s, 2H); 7.10-7.42 (m, 8H) |
| 7 | N-(2,6-diisopropyl- | p-toluidine | tetrahydrothio- | 2,6-diiso- | route 1 | 273 | (DMSO): 1.07-1.24 (12H, m), |
| | phenyl)-2-(2,4-dioxo- | | pyran-4-one | propyl-phenyl- | method 1 | | 1.69 (2H, td, J1 = 3.50 Hz; |
| | 1-p-tolyl-8-thia-1,3- | | | amine | | | J2 = 12.70 Hz); 2.31 (2H, d, |
| | diazaspiro[4.5]dec- | | | | | | J = 13.70 Hz); 2.36 (3H, s); |
| | 3-yl)-acetamide | | | | | | 2.52 (2H, d, J = 13.70 Hz); |
| | | | | | | | 3.07 (2H, m); 3.24 (2H, td, |
| | | | | | | | J1 = 3.50 Hz; J2 = 12.70 Hz); |
| | | | | | | | 4.30 (2H, s); 7.15 (2H, d, |
| | | | | | | | J = 8.10 Hz); 7.13-7.16 |
| | | | | | | | (2H, m); 7.24-7.28 (1H, m); |
| | | | | | | | 7.31 (2H, d, J = 8.10 Hz); |
| | | | | | | | 9.55 (1H, s) |
| 8 | 2-(8,8-difluoro- | p-toluidine | 4,4-difluoro- | 2,6-diiso- | route 1 | 263 | (DMSO): 1.07-1.13 (12H, m); |
| | 2,4-dioxo-1-p- | | cyclohexanone | propyl-phenyl- | method 1 | | 1.72 (2H, m); 2.00 (2H, m); |
| | tolyl-1,3-diaza- | | | amine | | | 2.14 (2H, m); 2.35 (3H, s); |
| | spiro[4.5]dec-3-yl)- | | | | | | 2.38-2.47 (2H, m); 3.07 |
| | N-(2,6-diisopropyl- | | | | | | (2H, m); 4.32 (2H, s); 7.14- |
| | phenyl)acetamide | | | | | | 7.16 (2H, m); 7.16 (2H, d, |
| | | | | | | | J = 8.10 Hz); 7.24-7.28 (1H, |
| | | | | | | | m); 7.31 (2H, d, J = 8.10 Hz); |
| | | | | | | | 9.56 (1H, s) |
| 9 | N-(2,6-diisopropyl- | p-toluidine | tetrahydro-4H- | 2,6-diiso- | route 1 | 296 | DMSO): 1.08-1.17 (12H, m); |
| | phenyl)-2-(2,4-dioxo- | | pyran-4-one | propyl-phenyl- | method 1 | | 1.71 (2H, bt, J = 13.50 Hz); |
| | 1-p-tolyl-8-oxa-1,3- | | | amine | | | 1.94 (2H, d, J = 13.50 Hz); |
| | diazaspiro-[4.5]dec-3- | | | | | | 2.37 (3H, s); 3.03-3.14 (2H, |
| | yl) acetamide | | | | | | m); 3.71-3.77 (2H, m); 3.91- |
| | | | | | | | 3.96 (2H, m); 4.32 (2H, s); |
| | | | | | | | 7.17 (2H, d, J = 8.00 Hz); |
| | | | | | | | 7.16-7.18 (2H, m); 7.25-7.26 |
| | | | | | | | (1H, m); 7.33 (2H, d, J = |
| | | | | | | | 8.00 Hz); 9.56 (1H, s) |
| 10 | N-(2,6-diisopropyl- | p-toluidine | tetrahydro- | 2,6-diiso- | route 1 | 294 | (DMSO): 1.07-1.14 (12H, m); |
| | phenyl)-2-(2,4,8,8- | | thiopyran-4-one | propyl-phenyl- | method 1 | | 2.13-2.19 (2H, m); 2.37 |
| | tetraoxo-1-p-tolyl- | | | amine | | | (3H, s); 2.47-2.50 (2H, m); |
| | 8lambda*6*-thia-1,3- | | | | | | 3.01-3.09 (2H, m); 3.19-3.22 |
| | diazaspiro[4.5]-dec- | | | | | | (2H, m); 3.56-3.63 (2H, m); |
| | 3-yl)-acetamide | | | | | | 4.34 (2H, s); 7.15-7.17 |
| | | | | | | | (2H, m); 7.20 (2H, d, J = |
| | | | | | | | 8.15 Hz); 7.24-7.28 (1H, m); |
| | | | | | | | 7.34 (2H, d, J = 8.15 Hz); |
| | | | | | | | 9.57 (1H, s) |
| 11 | N-(2,6-diethylphe- | p-toluidine | tetrahydro-4H- | 2,6-diethyl- | route 1 | 242 | (DMSO): 1.08 (6H, t, J = |
| | nyl)-2-(2,4-dioxo-1- | | pyran-4-one | phenyl-amine | method 1 | | 7.52 Hz) .1.65-1.73 (2H, m); |
| | p-tolyl-8-oxa-1,3- | | | | | | 1.91-1.94 (2H, m); 2.36 |
| | diaza-spiro-[4.5]dec-3- | | | | | | (3H, s); 2.46-2.54 (4H, m); |
| | yl) acetamide | | | | | | 3.71-3.75 (2H, m); 3.88-3.94 |
| | | | | | | | (2H, m); 4.30 (2H, s); 7.09 |
| | | | | | | | (2H, d, J = 8.00 Hz); 7.14- |
| | | | | | | | 7.16 (2H, m); 7.18-7.21 |
| | | | | | | | (1H, m); 7.31 (2H, d, J = |
| | | | | | | | 8.00 Hz); 9.56 (1H, s) |
| 12 | N-(2,6-diisopropyl- | 4-methoxy- | tetrahydro-4H- | 2,6-diiso- | route 2 | 232-234 | (DMSO): 1.08 (s, 6H); 1.11 |
| | phenyl)-2-[1-(4- | phenylamine | pyran-4-one | propyl-phe- | | | (s, 6H); 1.64-1.72 (m, 2H); |
| | methoxy-phenyl)-2,4- | | | nylamine | | | 1.90-1.93 (m, 2H); 3.03-3.10 |
| | dioxo-8-oxa-1,3-diaza- | | | | | | (m, 2H); 3.71-3.75 (m, 2H); |
| | spiro[4.5]-dec-3-yl]- | | | | | | 3.80 (s, 3H); 3.89-3.94 (m, |
| | acetamide. | | | | | | 2H); 4.30 (s, 2H); 7.03-7.06 |
| | | | | | | | (d, 2H); 7.14-7.19 (m, 4H); |
| | | | | | | | 7.24-7.27 (m, 1H); 9.53 (s, 1H) |
| 13 | N-(2,6-diisopropyl- | 3-methoxy- | tetrahydro-4H- | 2,6-diiso- | route 2 | 242-244 | (DMSO): 1.08 (s, 6H); 1.12 |
| | phenyl)-2-[1-(3- | phenylamine | pyran-4-one | propyl-phe- | | | (s, 6H); 1.70-1.78 (m, 2H); |
| | methoxy-phenyl)-2,4- | | | nylamine | | | 1.92-1.96 (m, 2H); 3.03-3.10 |
| | dioxo-8-oxa-1,3-diaza- | | | | | | (m, 2H); 3.72-3.76 (m, 2H); |
| | spiro[4.5]-dec-3-yl]- | | | | | | 3.78 (s, 3H); 3.89-3.95 (m, |
| | acetamide. | | | | | | 2H); 4.31 (s, 2H); 6.83-6.86 |
| | | | | | | | (m, 2H); 7.05-7.08 (dd, 1H, |
| | | | | | | | J = 2.04 Hz, J′ 5.96 Hz); |
| | | | | | | | 7.14-7.16 (d, 2H, J = 7.6 Hz); |
| | | | | | | | 7.24-7.27 (m, 1H); 7.39-7.44 |
| | | | | | | | (m, 1H); 9.54 (s, 1H) |
| 14 | N-(2,6-diisopropyl- | 4-methyl- | tetrahydro-4H- | 2,6-diiso- | route 1 | — | (DMSO): 1.1 (m, 6H); 1.66- |
| | phenyl)-2-[1-(4-meth- | sulfanyl- | pyran-4-one | propyl-phe- | method 2 | | 1.74 (m, 1H); 0.9 (d, 1H); |
| | ylsulfanyl-phenyl)-2,4- | phenylamine | | nylamine | | | 3.06 (m, 1H); 3.3 (s, 3H); |
| | dioxo-8-oxa-1,3-diaza- | | | | | | 3.7 (m, 1H); 3.9 (t, 1H); |
| | spiro [4.5]-dec-3-yl]- | | | | | | 4.3 (s, 1H); 7.1 (d, 1H); |
| | acetamide | | | | | | 7.2 (d, 1H); 7.25 (t, 1H); |
| | | | | | | | 7.37 (d, 1H) |

All the NMR (nuclear magnetic resonance) spectra are in accordance with the proposed structures. The chemical shifts are expressed in parts per million. The internal reference is tetramethylsilane. The following abbreviations are used: CDCl₃ = deuterated chloroform, DMSO = deuterated dimethyl sulfoxide

### Example 15

### Biological Tests

The compounds of formula (I) according to the invention were subjected to a test for evaluating their inhibitory activity towards the enzyme ACAT-1, inspired by the following publication: “Identification of ACAT1- and ACAT2-specific inhibitors using a novel, cell based fluorescence assay: individual ACAT uniqueness”, J. Lipid. Res. (2004) vol. 45, pages 378-386.

The principle of this test is based on the use of NBD-cholesterol, a cholesterol analogue whose fluorescence depends on its environment. When this molecule is in a polar environment, it is weakly fluorescent, whereas in a non-polar environment, it is strongly fluorescent. Free NBD-cholesterol becomes inserted in cell membranes and is weakly fluorescent in this polar environment. When NBD-cholesterol is esterified with ACAT, the NBD-cholesterol ester enters non-polar lipid droplets and is then strongly fluorescent.

The method below is applied: HepG2 cells are incubated in the presence of NBD-cholesterol (1 μg/ml) and of the test compound of formula (I) in black transparent-bottomed 96-well plates, at a rate of 30 000 cells per well. After incubation for 6 hours at 37° C. under 5% CO₂, the medium is removed by turning upside-down and the cells are washed with twice 100 μl of PBS. After addition of 50 μl of lysis buffer (10 mM NaPO₄, 1% Igepal), the plates are shaken for 5 minutes and the fluorescence is read (excitation at 490 nm, emission at 540 nm) on a Fusion machine (Perkin-Elmer). By way of illustration, an IC_{H} of 1600 nM is obtained for compound (1), an IC₅₀ of 29 nM is obtained for compound (2), an IC₅₀ of 86 nM is obtained for compound (3), an IC₅₀ of 5.8 nM is obtained for compound (4), an IC₅₀ of 20 nM is obtained for compound (5), an IC₅₀ of 6.4 nM is obtained for compound (6), an IC₅₀ of 1.8 nM is obtained for compound (7), an IC₅₀ of 1.5 nM is obtained for compound (8), an IC₅₀ of 0.4 nM is obtained for compound (9) and an IC₅₀ of 9.4 nM is obtained for compound (11).

### Example 16

### Formulations

According to another of its aspects, the invention relates to compositions containing a compound of general formula (I) as described above, or a pharmaceutically acceptable salt, or a hydrate or solvate of the said compound, and also at least one pharmaceutically acceptable excipient. Various formulations containing the compounds according to the invention are given below.

A—Oral Route

### (a) 0.2 g Tablet

| | |
|---|---|
| Compound 1 | 0.01 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

### (b) Drinkable Suspension in 5 ml Vials

| Compound 2 | 0.001 | g |
|---|---|---|
| Glycerol | 0.500 | g |
| 70% Sorbitol | 0.500 | g |
| Sodium saccharinate | 0.010 | g |
| Methyl para-hydroxybenzoate | 0.040 | g |
| Flavouring | qs | |
| Purified water | qs 5 | ml |

B—Topical Route

### (a) Ointment

| | |
|---|---|
| Compound 6 | 0.300 g |
| White petroleum jelly codex | qs 100 g |

### (d) Lotion

| | |
|---|---|
| Compound 4 | 0.100 g |
| Polyethylene glycol (PEG 400) | 69.900 g |
| 95% Ethanol | 30.000 g |

### (e) Hydrophobic Ointment

| | |
|---|---|
| Compound 9 | 0.300 g |
| Isopropyl myristate | 36.400 g |
| Silicone oil (Rhodorsil 47 V 300) | 36.400 g |
| Beeswax | 13.600 g |
| Silicone oil (Abil 300 000 cSt) | qs 100 g |

### (f) Nonionic Oil-in-Water Cream

| | |
|---|---|
| Compound 8 | 1.000 g |
| Cetyl alcohol | 4.000 g |
| Glyceryl monostearate | 2.500 g |
| PEG 50 stearate | 2.500 g |
| Shea butter | 9.200 g |
| Propylene glycol | 2.000 g |
| Methyl para-hydroxybenzoate | 0.075 g |
| Propyl para-hydroxybenzoate | 0.075 g |
| Sterile demineralized water | qs 100 g |

1. A compound of formula (I): 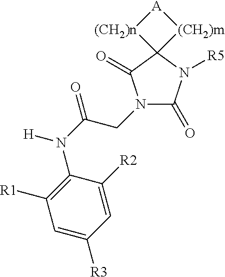
in which:
R₁ represents a group C₁₋₆ alkyl, C₃₋₇ cycloalkyl, C₁₋₆ alkyloxy, C₁₋₆ fluoroalkyl, C₁₋₆ fluoroalkyloxy or a group —(CH₂)ₙ—C₃₋₇ cycloalkyl,
R₂ and R₃ are identical or different and represent a hydrogen, chlorine, fluorine, bromine or iodine atom or a group C₁₋₆ alkyl, C₃₋₆ cycloalkyl, C₁₋₆ alkylthio, C₁₋₆ alkyloxy, C₁₋₆ fluoroalkyl, C₁₋₆ fluoroalkyloxy or a group —(CH₂)ₙ—C₃₋₇ cycloalkyl,
R₅ represents a group selected from the group consisting of:
an unsubstituted phenyl group or a phenyl group substituted with one, two or three identical or different substituents selected from the group consisting of fluorine, chlorine and bromine atoms and groups C₁₋₄ alkyl, C₁₋₄ alkylthio, trifluoromethyl, hydroxymethyl, mono-, di- and tri-fluoromethoxy, C₁₋₄ alkyloxy, phenoxy, benzyloxy, phenyl, 2-pyridyl, 3-pyridyl and 4-pyridyl,
a linear or branched group C₂₋₁₂ alkyl, optionally substituted with one or more hydroxyl groups or fluorine atoms,
a group C₃₋₁₂ cycloalkyl or a group —(CH₂)ₚ—C₂₋₁₂ cycloalkyl,
a group —(CH₂)ₙ-aryl in which n is equal to 1, 2 or 3, optionally substituted with one or more groups Rₐ,
A represents:
either an oxygen atom,
or a group S(O)ₚ,
or a group NR_{b},
or a carbon atom substituted with one or two fluorine atoms or a hydroxyl group,
Rb is selected from the group consisting of: H, C₁₋₆ alkyl, C(O)C₁₋₆ alkyl, C(O)aryl, C(O)—(CH₂)ₚ-aryl, the aryl groups mentioned being optionally substituted with one or more groups Ra,
Rₐ represents either a hydrogen, fluorine or chlorine atom or a group C₁₋₆ alkyl, C₃₋₇ cycloalkyl, C₁₋₆ alkyloxy, C₁₋₆ alkylthio, C₁₋₆ fluoroalkyl, C₁₋₆ fluoroalkyloxy, OH, CH₂OH, COORc or CN,
R_{c} represents a group C₁₋₆ alkyl, C₃₋₇ cycloalkyl or —(CH₂)ₙ—C₃₋₇ cycloalkyl,
m and n each represents an integer, the sum of which ranges from 3 to 6,
p represents 0, 1 or 2,
and also pharmaceutically acceptable salts, solvates or hydrates thereof and conformers or rotamers thereof.

2. The compound according to claim 1, wherein the compound has one or a combination of the following characteristics:
R₁ represents a methyl, ethyl or isopropyl group,
R₂ represents a chlorine or bromine atom or a methyl, ethyl, isopropyl or tert-butyl group,
R₃ represents a hydrogen atom,
n=m=2,
A represents either an oxygen atom or a carbon atom substituted with a group —OH,
R₅ is selected from the group consisting of:
an unsubstituted phenyl group or a phenyl group substituted with one, two or three identical or different substituents selected from the group consisting of fluorine, chlorine and bromine atoms and methyl, ethyl, n-butyl, trifluoromethyl, hydroxymethyl, di- and tri-fluoromethoxy, methoxy, phenoxy and benzyloxy groups,
a group C₂₋₁₂ alkyl, optionally substituted with one or more hydroxyl groups or fluorine atoms,
a sec-butyl, n-propyl, n-butyl, n-pentyl, 2,2-dimethylpropyl, n-hexyl, n-heptyl, n-octyl or n-nonyl group, an n-butyl group substituted in position 4 with three fluorine atoms, an n-propyl group substituted in position 3 with three fluorine atoms, an n-butyl substituted in position 4 with a hydroxyl group, or an n-propyl group substituted in position 3 with a hydroxyl group,
a group —CH₂-cyclopropyl, —CH₂-cyclohexyl, cyclopentyl, cyclohexyl or cycloheptyl, and
a group —(CH₂)ₙ-aryl in which n is equal to 1 or 2 and the aryl group may can be optionally substituted with a group Rₐ, optionally in the meta or para position, with a methyl, trifluoromethyl or methoxy group or a fluorine atom.

3. The compound according to claim 1, wherein the compound has one or a combination of the following characteristics, which are not mutually exclusive:
R₁═R₂═=iPr, R₃═H,
R₁═R₂=Et, R₃═H,
n=m=2,
A represents either an oxygen atom or a carbon atom substituted with a group —OH,
R₅ represents an unsubstituted phenyl group or a phenyl group substituted, in the meta or para position, with a chlorine or fluorine atom or a methyl or methoxy group.

4. The compound according to claim 1, wherein the compound is selected from the group of compounds below, and pharmaceutically acceptable salts, solvates, hydrates, conformers and rotamers thereof consisting of:
2-(8-acetyl-2,4-dioxo-1-p-tolyl-1,3,8-triazaspiro[4.5]dec-3-yl)-N-(2,6-diisopropylphenyl)acetamide;
3-[(2,6-diisopropylphenylcarbamoyl)methyl]-2,4-dioxo-1-p-tolyl-1,3,8-triazaspiro[4.5]decane-8-carboxylic acid tert-butyl ester;
2-(8-benzoyl-2,4-dioxo-1-p-tolyl-1,3,8-triazaspiro[4.5]dec-3-yl)-N-(2,6-diisopropylphenyl)acetamide;
2-(8-benzyl-2,4-dioxo-1-p-tolyl-1,3,8-triazaspiro[4.5]dec-3-yl)-N-(2,6-diisopropylphenyl)acetamide;
N-(2,6-diisopropylphenyl)-2-(8-methyl-2,4-dioxo-1-p-tolyl-1,3,8-triazaspiro[4.5]dec-3-yl)acetamide;
N-(2,6-diisopropylphenyl)-2-(8-hydroxy-2,4-dioxo-1-p-tolyl-1,3-diazaspiro[4.5]dec-3-yl)acetamide;
N-(2,6-diisopropylphenyl)-2-(2,4-dioxo-1-p-tolyl-8-thia-1,3-diazaspiro[4.5]dec-3-yl)acetamide;
2-(8,8-difluoro-2,4-dioxo-1-p-tolyl-1,3-diazaspiro[4.5]dec-3-yl)-N-(2,6-diisopropylphenyl)acetamide;
N-(2,6-diisopropylphenyl)-2-(2,4-dioxo-1-p-tolyl-8-oxa-1,3-diazaspiro[4.5]dec-3-yl)acetamide;
N-(2,6-diisopropylphenyl)-2-(2,4,8,8-tetraoxo-1-p-tolyl-8lambda*6*-thia-1,3-diazaspiro[4.5]dec-3-yl)acetamide;
N-(2,6-diethylphenyl)-2-(2,4-dioxo-1-p-tolyl-8-oxa-1,3-diazaspiro[4.5]dec-3-yl)acetamide;
N-(2,6-diisopropylphenyl)-2-[1-(4-methoxyphenyl)-2,4-dioxo-8-oxa-1,3-diazaspiro[4.5]dec-3-yl]acetamide;
N-(2,6-diisopropylphenyl)-2-[1-(3-methoxyphenyl)-2,4-dioxo-8-oxa-1,3-diazaspiro[4.5]dec-3-yl]acetamide; and
N-(2,6-diisopropylphenyl)-2-[1-(4-methylsulfanylphenyl)-2,4-dioxo-8-oxa-1,3-diazaspiro[4.5]dec-3-yl]acetamide.

5. The compound according to claim 1, wherein the compound is provided in the form of a medicament.

6. A pharmaceutical composition comprising, in a physiologically acceptable support, at least one compound according to claim 1.

7. The composition according to claim 6, wherein the concentration of the compound according to claim 1 is between 0.001% and 10% by weight relative to the total weight of the composition.

8. The composition according to claim 7, wherein the concentration of the compound(s) according to claim 1 is between 0.01% and 5% by weight relative to the total weight of the composition.

9. A cosmetic composition, wherein the composition comprises, in a physiologically acceptable support, at least one compound according to claim 1.

10. The composition according to claim 1, wherein the composition is in a form suitable for topical application.

11. The composition according to claim 10, wherein the composition is in the form of a cream, a milk, a lotion, a gel, an ointment, a pomade, a suspension of microspheres or nanospheres or lipid or polymer vesicles, an impregnated padspad, solutions a solution, sprays a spray, a mousse, a stick, a soap, a shampoo or a washing base.

12. A cosmetic method, the method comprising administering a composition as defined in claim 10 to an individual subject in need thereof for body or hair hygiene.

13. A method of making a medicament, the method comprising making the medicament so that it comprises an effective amount of a compound according to claim 1, for treating an indication selected from the group consisting of a sebaceous gland disorder, an ocular pathology, hypercholesterolaemia, arteriosclerosis and Alzheimer's disease.

14. A method of making a medicament, the method comprising making the medicament so that it comprises an effective amount of a compound according to claim 1, for treating acne.

15. The method according to claim 13, wherein the sebaceous gland disorder is selected from the group consisting of hyperseborrhoea, acne, seborrhoeic dermatitis and atopic dermatitis.

16. The method according to claim 13, wherein the ocular pathology is blepharitis or meibomitis.
